(19) United States
(12) Patent Application Publication
(10) Pub. No.: US 2019/0269300 A1
(43) Pub. Date: Sep. 5, 2019

(54) BENDING OPERATION MECHANISM OF ENDOSCOPE

(71) Applicant: OLYMPUS CORPORATION, Tokyo (JP)

(72) Inventors: Toshihiro MATSUI, Tokyo (JP); Koji YASUNAGA, Tokyo (JP); Yuta SATO, Tokyo (JP)

(73) Assignee: OLYMPUS CORPORATION, Tokyo (JP)

(21) Appl. No.: 16/415,364

(22) Filed: May 17, 2019

Related U.S. Application Data
(63) Continuation of application No. PCT/JP2017/036919, filed on Oct. 12, 2017.

(30) Foreign Application Priority Data
Jan. 5, 2017 (JP) 2017-000645

(51) Int. Cl. A61B 1/005

(52) U.S. Cl. CPC A61B 1/0052

(57) ABSTRACT

An endoscope bending operation mechanism includes: an operation lever with one end being rotatably held; a frame configured to rotate with the operation lever; a fixing member rotatably supporting the frame; a rotation shaft coupled with an end portion of one of the frame and the fixing member; a bearing portion provided on the other of the frame and the fixing member and having a hole rotatably holding the rotation shaft; and a position defining portion configured to abut a part of the other of the frame and the fixing member to define a position of the rotation shaft. The position of the rotation shaft is adjustable, and by adjusting the position of the rotation shaft, a clearance between the bearing portion and the position defining portion in a direction along a central axis is adjusted.

### CROSS REFERENCE TO RELATED APPLICATION

This application is a continuation application of PCT/JP2017/036919 filed on Oct. 12, 2017 and claims benefit of Japanese Application No. 2017-000645 filed in Japan on Jan. 5, 2017, the entire contents of which are incorporated herein by this reference.

### BACKGROUND OF INVENTION

### 1. Field of the Invention

The present invention relates to an endoscope bending operation mechanism provided with a joystick type operation member for a bending operation.

### 2. Description of the Related Art

Conventionally, endoscopes configured having an insertion portion in an elongated tubular shape have been widely used, for example, in a medical field, an industrial field and the like. Among the endoscopes, a medical endoscope used in the medical field is configured to make it possible to observe organs and the like by inserting an insertion portion, for example, into a body cavity of a living body and perform various treatments to the organs and the like using a treatment instrument inserted into a treatment instrument insertion channel provided in the endoscope, as needed. An industrial endoscope used in the industrial field is configured to make it possible to, by inserting an insertion portion, for example, into an inside of a jet engine, a device such as factory piping, mechanical equipment or the like, observe and inspect a state of scratches, corrosion and the like inside the device or the like.

The insertion portion of the conventional endoscope of the kind is commonly configured in a form of providing a distal end rigid portion, a bending portion and an elongated tubular member (a flexible tube having flexibility or a rigid tube configured with a rigid member made of metal or the like) being connected in that order from a distal end side. Among the above components, the bending portion is a part configured to be bendable relative to an insertion axis by operating an operation member provided on the operation portion provided being connected to a proximal end of the insertion portion. The conventional endoscope is configured by providing a bending operation mechanism inside the operation portion and the insertion portion to cause a bending operation of the bending portion to be realized.

The endoscope bending operation mechanism is configured with the operation member for a bending operation provided on the operation portion, a bending wire configured to transmit an operation input of the operation member to the bending portion on the distal end side of the insertion portion, a bending mechanism portion interposed between the above operation member and the above bending wire, and the like. Among the above components, as the operation member for a bending operation, for example, a rotating operation type operation member is common. Additionally, there is, for example, a joystick type operation member in a form of causing a stick-shaped member to tilt.

As for the bending operation mechanism using the above joystick type operation member as the operation member for a bending operation in an endoscope, bending operation mechanisms in various forms have been conventionally proposed, for example, by Japanese Patent Application Laid-Open Publication No. H6-169883, Japanese Patent Application Laid-Open Publication No. 2011-242607 and the like.

In the conventional endoscope bending operation mechanisms using the joystick type operation member, a proximal end portion of the stick-shaped member is held such that, relative to a predetermined central axis, the proximal end portion is rotatable around the central axis. In this case, the proximal end portion of the stick-shaped member rotates around the central axis by receiving rotation shafts arranged coaxially with the central axis by bearing portions provided on a fixing member. According to such a configuration, the stick-shaped member is configured to, when the stick-shaped member is caused to tilt, cause the rotation shafts to rotate in the bearing portions and rotate around the central axis.

### SUMMARY OF THE INVENTION

An endoscope bending operation mechanism of one aspect of the present invention is provided with: an operation lever with one end being rotatably held relative to a predetermined central axis; a frame with which the operation lever is coupled, the frame being configured to rotate relative to the predetermined central axis with the operation lever; a fixing member rotatably supporting the frame; a rotation shaft coupled with an end portion of one of the frame and the fixing member coaxially with the predetermined central axis; a bearing portion provided on the other of the frame and the fixing member and having a hole rotatably holding the rotation shaft; and a position defining portion provided on a part of the rotation shaft, the position defining portion having an outer diameter portion larger than an inner diameter of the hole and abutting a part of the other of the frame and the fixing member to define a position of the rotation shaft in an axial direction of the rotation shaft. The rotation shaft is configured such that a position in a direction along the predetermined central axis is adjustable, and by adjusting the position of the rotation shaft in the direction along the predetermined central axis, a clearance between the bearing portion and the position defining portion in the direction along the predetermined central axis is adjusted.

An endoscope bending operation mechanism of the second aspect of the present invention is provided with: an operation lever with one end being rotatably held relative to a predetermined central axis; a frame configured to rotatably support the operation lever; a rotation shaft coupled with one of the frame and the operation lever coaxially with the predetermined central axis; a bearing portion provided on the other of the frame and the operation lever and having a hole rotatably holding the rotation shaft; and a position defining portion provided on a part of the rotation shaft, the position defining portion having an outer diameter portion larger than an inner diameter of the hole and abutting a part of the other of the frame and the operation lever to define a position of the rotation shaft in an axial direction of the rotation shaft. The rotation shaft is configured such that a position in a direction along the predetermined central axis is adjustable, and by adjusting the position of the rotation shaft in the direction along the predetermined central axis, a clearance between the bearing portion and the position defining portion in the direction along the predetermined central axis is adjusted.

### BRIEF DESCRIPTION OF THE DRAWINGS

FIG. 1 shows a schematic configuration of a whole endoscope system including an endoscope provided with a bending operation mechanism of one embodiment of the present invention;

FIG. 2 shows the endoscope bending operation mechanism of the one embodiment of the present invention and is a main part enlarged perspective view showing an internal configuration of the bending operation mechanism;

FIG. 3 is a longitudinal cross-sectional view of a plane along a [3]-[3] line in FIG. 2;

FIG. 4 is a main part enlarged cross-sectional view enlargingly showing an area near a rotation shaft indicated by an arrow symbol [4] in FIG. 3;

FIG. 5 is a conceptual diagram showing a state of contact between a spherical portion of a first rotation shaft and an inner circumference of a hole of a first bearing portion in the endoscope bending operation mechanism in FIG. 2 and is a diagram illustrating a case where the hole of the first bearing portion is coaxially formed along a central axis;

FIG. 6 is a conceptual diagram showing a state of contact between the spherical portion of the first rotation shaft and the inner circumference of the hole of the first bearing portion in the endoscope bending operation mechanism in FIG. 2 and is a diagram illustrating a case where the hole of the first bearing portion is formed being slightly displaced from the central axis;

FIG. 7 is a main part enlarged cross-sectional view showing a first modification of a position adjustment mechanism in the endoscope bending operation mechanism of the one embodiment of the present invention;

FIG. 8 is a main part enlarged cross-sectional view showing a second modification of the position adjustment mechanism in the endoscope bending operation mechanism of the one embodiment of the present invention; and

FIG. 9 is a main part enlarged cross-sectional view showing a third modification of the position adjustment mechanism in the endoscope bending operation mechanism of the one embodiment of the present invention.

### DETAILED DESCRIPTION OF THE PREFERRED EMBODIMENT(S)

The present invention will be described below by an embodiment shown in drawings. Each of the drawings used in the description below is schematic, and a dimensional relationship, reduce scale and the like of each member may be shown different for each component in order to show the component in a recognizable size on the drawing. Therefore, in terms of the number for each component, a shape of each component, a ratio of sizes among respective components, relative positional relationships among the respective components and the like illustrated in each of the drawings, the present invention is not limited to a form shown in the drawing.

### One Embodiment

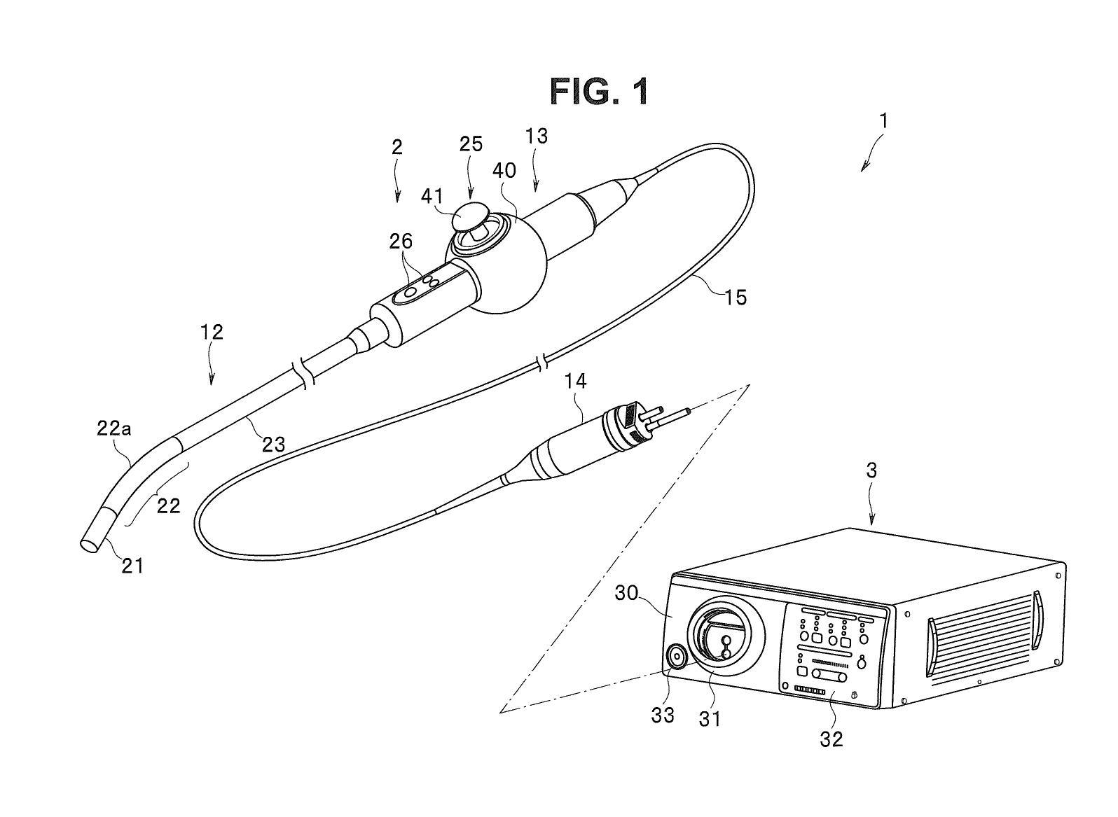

FIG. 1 is a diagram showing a schematic configuration of a whole endoscope system including an endoscope provided with a bending operation mechanism of one embodiment of the present invention.

First, before describing details of the bending operation mechanism of the present embodiment, the schematic configuration of the endoscope system including the endoscope provided with the bending operation mechanism will be described below using FIG. 1.

As shown in FIG. 1, an endoscope system 1 is a medical apparatus that is mainly configured with an endoscope 2 and a camera control unit 3.

The camera control unit 3 is a control device configured to control the endoscope 2 and is provided with an image processing device and a light source device. In other words, the camera control unit 3 includes a control device including a control circuit and the like configured to control an image pickup unit (not shown) and the like provided in the endoscope 2, an image processing device including an image processing circuit and the like configured to receive an image signal acquired by the above image pickup unit (not shown) of the endoscope 2 and perform various kinds of image processing and the like, a light source device including a light source (a halogen lamp or the like; not shown) configured to supply illumination light to the endoscope 2, and the like.

On a front of the camera control unit 3, an operation panel 30 provided with various kinds of operation members and the like is provided. The operation panel 30 is provided with a receptacle portion 31 which is a connection portion configured to connect to the endoscope 2, an operation/display portion 32 on which an operation member for performing various kinds of operations, a display member for displaying states, and the like are arranged, a power source switch 33 and the like.

Note that an endoscope connector 14 of the endoscope 2 to be described later is connected to the above receptacle portion 31. Consequently, electrical connection between the camera control unit 3 and the endoscope 2 is secured.

The endoscope 2 is mainly configured having an elongated insertion portion 12, an operation portion 13 provided being connected to a proximal end of the insertion portion 12, an endoscope connector 14 connected to the receptacle portion 31 of the camera control unit 3, and the like.

The insertion portion 12 is configured having a distal end portion 21 mainly formed with a member made of metal such as stainless steel, a flexibly bendable bending portion 22 and a tubular member 23 such as an elongated rigid tube which is formed by a tube made of metal such as stainless steel, or a flexible tube having flexibility, which are connectedly provided in that order from a distal end side.

The distal end portion 21 includes an image pickup portion (not shown) using a CCD sensor, a CMOS sensor or the like. From the image pickup portion, a communication cable for drive control, optical transmission fibers for high-speed transmission to transmit an image pickup signal, and the like are extended, and inserted inside the above insertion portion 12.

Inside the bending portion 22, a plurality of bending pieces (not shown) are arranged in line in a longitudinal direction. The plurality of bending pieces are configured to, by being mutually rotated by a plurality of (for example, four) bending operation wires (not shown) being pulled or released, be able to cause the bending portion 22 to bend in an arbitrary direction. The above bending portion 22 is provided with bending rubber 22a, which is an outer cover covering the plurality of bending pieces, in a manner of covering an outer surface.

Inside the tubular member 23, the communication cable and the optical transmission fibers extended from the image pickup portion of the distal end portion 21, a light guide for transmitting illumination light to the distal end portion 21 and the like are inserted from the distal end portion 21 via the bending portion 22. Inside the tubular member 23, furthermore, a plurality of bending operation wires (not shown) extended to a proximal end side inside the tubular member 23, with distal ends being connected to a most distal bending piece (not shown) of the bending portion 22, are inserted.

The operation portion 13 is a component unit provided being connected to the proximal end of the insertion portion 12 and configured with a case that is configured having an internal space. The operation portion 13 is provided with a bending operation mechanism 25 (to be described in detail later) for remotely operating the bending portion 22 via the bending operation wires and various kinds of switches 26 and the like for operating the camera control unit 3 and the like.

Further, a flexible cable 15 (a universal cord) is extended from the operation portion 13. The above endoscope connector 14 is provided being connected to a distal end of the flexible cable 15. Inside the operation portion 13, the above communication cable, the optical transmission fibers, the light guide and the like extended from the insertion portion 12 are inserted. The various kinds of internal components are inserted inside the flexible cable 15 and connected to the endoscope connector 14. Due to such a configuration, when the endoscope connector 14 is connected to the receptacle portion 31, the operation portion 13 of the endoscope 2 and the endoscope connector 14 are connected via the flexible cable 15.

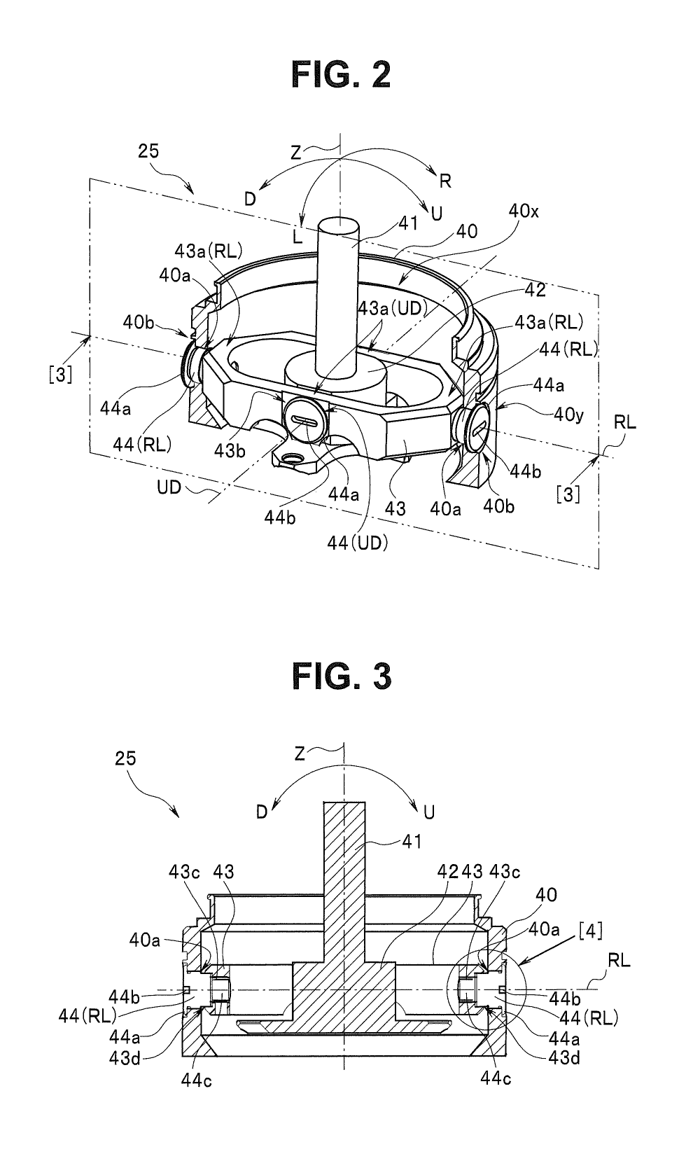

Next, a configuration of the endoscope bending operation mechanism of the present embodiment will be described below in detail, using mainly FIGS. 2 to 4.

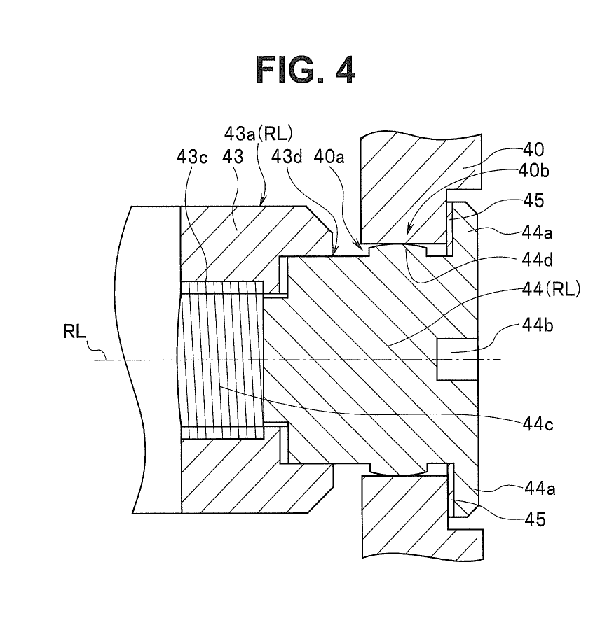

FIGS. 2 and 3 are diagrams showing the endoscope bending operation mechanism of the one embodiment of the present invention. FIG. 2 is a main part enlarged perspective view showing an internal configuration of the endoscope bending operation mechanism of the present embodiment. FIG. 3 is a longitudinal cross-sectional view of a plane along a [3]-[3] line in FIG. 2. FIG. 4 is a main part enlarged cross-sectional view enlargingly showing an area near a rotation shaft indicated by an arrow symbol [4] in FIG. 3.

As shown in FIGS. 2 and 3, the endoscope bending operation mechanism 25 of the present embodiment is configured with a casing 40, a bending operation lever 41 which is an operation lever, a frame 43, a plurality of rotation shafts and the like.

The casing 40 is a case in which the respective component members of the bending operation mechanism 25 are disposed and is an exterior member. The casing 40 is also a fixing member supporting the bending operation lever 41 rotatably around a predetermined central axis (to be described in detail later).

Note that in the present embodiment, an example in which a part of an exterior case of the operation portion 13 is integrally formed as the casing 40 of the bending operation mechanism 25. However, a configuration of the above casing 40 is not limited to the example. For example, a form is also possible in which the casing 40 is in a form of being configured separately from the operation portion 13, and the casing unit as a separate body is fixed to the above operation portion 13.

Here, the predetermined central axis is an axis to be a center of rotation at the time of causing the bending operation lever 41 to tilt. In the present embodiment, the above predetermined central axis is assumed to be a plurality of axes extending in a direction orthogonal to an axial direction of the bending operation lever 41 itself (a direction along a two-dot chain line indicated by reference character Z in FIG. 2) as shown in FIG. 2. In other words, in the present embodiment, the above predetermined central axis refers to two two-dot chain lines indicated by reference character RL and reference character UD in FIG. 2. Hereinafter, the predetermined central axes will be referred to as a central axis RL and a central axis UD.

The above central axis RL and the above central axis UD are set to be orthogonal to each other. When the above bending operation lever 41 is caused to tilt in a direction of an arrow R or a direction of an arrow L shown in FIG. 2, the bending operation lever 41 rotates around the above central axis RL. When the bending operation lever 41 is caused to tilt in a direction of an arrow U or a direction of an arrow D shown in FIG. 2, the bending operation lever 41 rotates around the above central axis UD.

Note that though the example in which two predetermined central axes (RL, UD) to be centers of rotation of the bending operation lever 41 are provided is shown in the present embodiment, the number of predetermined central axes is not limited to the form. For example, a configuration can be made in which the number of predetermined central axes described above is one. In this case, the operation of tilting the bending operation lever 41 is, for example, either an operation only in a direction of the arrow R or L or an operation only in a direction of the arrow U or D.

The bending operation lever 41 is an operation member for performing a bending operation by causing the bending operation lever 41 to tilt. The bending operation lever 41 is configured with a stick-shaped member, and a lever base 42 is formed on one end of the stick-shaped member. The lever base 42 is held in (second end portions 43a (UD) of) the frame 43 so that, relative to the predetermined central axis UD, one end (the lever base 42) is rotatable around the central axis UD.

The frame 43 is a lever holding member with which the lever base 42 of the bending operation lever 41 is coupled and which is configured to hold the bending operation lever 41 rotatably around the predetermined central axis UD and rotate relative to the predetermined central axis RL together with the bending operation lever 41.

In other words, the frame 43 rotatably supports the lever base 42 of the above bending operation lever 41 relative to one (the central axis UD) of the above predetermined central axes (RL, UD). Further, the frame 43 is rotatably supported relative to a part (a first bearing portion 40b; to be described later) of the above casing 40 relative to the other (the central axis RL) of the above predetermined central axes (RL, UD).

The plurality of rotation shafts include two first rotation shafts 44 (RL) disposed at predetermined parts (first bearing portions 40b; to be described later) of the casing 40 and two second rotation shafts 44 (UD) (see FIG. 2) disposed at predetermined parts (second bearing portions 43b; to be described later) of the frame 43.

The above first rotation shafts 44 (RL) are shaft members arranged coaxially with one central axis RL and configured to rotatably support two first end portions 43a (RL) of the frame 43 relative to parts (the first bearing portions 40b; to be described later) of the casing 40, respectively.

The above second rotation shafts 44 (UD) are shaft members arranged coaxially with the other central axis UD and configured to rotatably support the lever base 42 of the bending operation lever 41 relative to the two second end portions 43a (UD) of the frame 43.

Note that all of the plurality of rotation shafts are formed in similar forms though parts where the rotation shafts are disposed and component members targeted by actions of the rotation shafts are different, which will be described in detail later.

The following is a detailed configuration of the endoscope bending operation mechanism 25 of the present embodiment.

The above casing 40 is provided with the plurality of (two) first bearing portions 40b having a plurality of (two) holes 40a rotatably holding the two first rotation shafts 44 (RL), respectively, among the above plurality of rotation shafts. Here, the above plurality of (two) first bearing portions 40b are formed integrally with the casing 40 as a part of the casing 40.

An opening 40x (see FIG. 2) is formed on the above casing 40. The opening 40x is a cavity portion configured to restrict a movable area when a tilting operation of the bending operation lever 41 is performed. Therefore, the above opening 40x is open in a direction in which the above bending operation lever 41 protrudes from an exterior surface of the above operation portion 13 when the bending operation mechanism 25 is incorporated in the above operation portion 13. On the casing 40, a side wall 40y (see FIG. 2) is formed such that the side wall 40y surrounds a periphery portion of the above opening 40x. The above bending operation lever 41 is disposed in an internal area of the opening 40x.

On the casing 40, the above plurality of (two) first bearing portions 40b are provided at mutually facing positions on the above side wall 40y, and the plurality of (two) holes 40a are provided at mutually facing positions on the side wall 40y. Here, the plurality of (two) holes 40a of the plurality of (two) first bearing portions 40b are formed such that an axis connecting centers of the respective holes 40a is coaxial with one (the central axis RL) of the above predetermined central axes.

On the above casing 40, the first rotation shafts 44 (RL) are insertedly arranged in the above plurality of (two) holes 40a, respectively. The first rotation shafts 44 (RL) support the frame 43 rotatably around the central axis RL in the first bearing portions 40b of the casing 40. In this case, at least parts of the first rotation shafts 44 (RL) that are in contact with inner circumferences of the holes 40a are spherically formed. The parts are referred to as spherical portions 44d (see FIG. 4).

Since the parts of the first rotation shafts 44 (RL) that are in contact with the inner circumferences of the holes 40a are provided with the spherical portions 44d, the spherical portions 44d of the first rotation shafts 44 (RL) are in line contact with the inner circumferences of the holes 40a of the first bearing portion 40b, respectively.

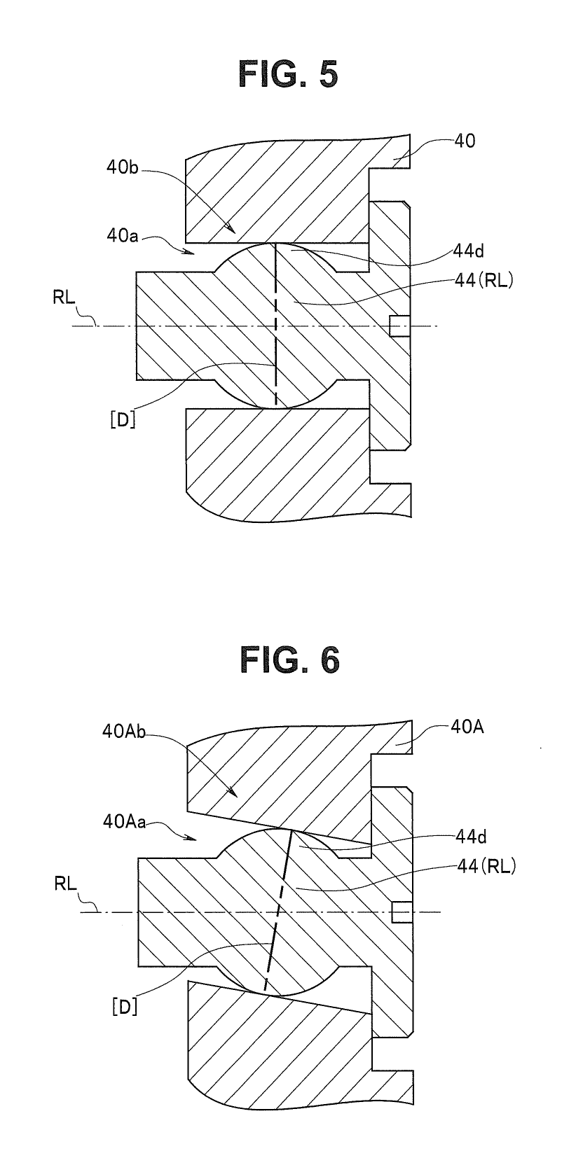

Here, FIGS. 5 and 6 are conceptual diagrams showing a state of contact between the spherical portion of the first rotation shaft and the inner circumference of the hole of the first bearing portion. FIG. 5 is a diagram illustrating a case where the hole 40a of the first bearing portion 40b is coaxially formed along the central axis RL. FIG. 6 is a diagram illustrating a case where a hole 40Aa of a first bearing portion 40Ab is formed being slightly displaced from the central axis RL.

In FIGS. 5 and 6, a two-dot chain line indicated by a reference character [D] indicates a part where a part of the spherical portion 44d is in line contact with an inner circumference of the hole 40a, 40Aa.

In a normal case, the hole 40a of the first bearing portion 40b of the casing 40 is coaxially formed along the central axis RL as shown in FIG. 5. In this case, since the spherical portion 44d of the first rotation shaft 44 (RL) is in line contact (see reference character [D]) with the inner circumference of the hole 40a of the first bearing portion 40b, smooth rotation is secured.

On the other hand, according to accuracy of machining, the hole 40Aa of the first bearing portion 40Ab of a casing 40A may be formed being slightly displaced from the central axis RL as shown in FIG. 6 even if the accuracy is within an allowable tolerance range. Even in such a case, since line contact between the spherical portion 44d of the first rotation shaft 44 (RL) and the inner circumference of the hole 40Aa of the first bearing portion 40Ab (see reference character [D]) is secured, smooth rotation is secured.

Note that though the example in which the above spherical portion 44d is formed on the first rotation shaft 44 (RL) is shown in the present embodiment, a place where the spherical portion 44d is formed is not limited to the example. For example, a form is also possible in which the above spherical portion is formed on at least a part on the inner circumference of the hole that is in contact with an outer circumference of the rotation shaft.

A screw portion 44c is formed on a part of the first rotation shaft 44 (RL) near a distal end of the first rotation shaft 44 (RL) as shown in FIG. 4. In a part (the first end portion 43a (RL)) of the frame 43, a hole 43d is formed coaxially with the central axis RL in a manner of causing the hole 43d to correspond to the screw portion 44c as shown in FIG. 4. For the hole 43d, a screw groove 43c to screw onto the above screw portion 44c is formed coaxially with the central axis RL. According to the configuration, the two first rotation shafts 44 (RL) are insertedly arranged in the plurality of (two) holes 40a, respectively, on the casing 40, and the respective screw portions 44c of the two first rotation shafts 44 (RL) screw into the screw grooves 43c of the frame 43. Consequently, the frame 43 is supported rotatably around the central axis RL by the two first rotation shafts 44 (RL) in the first bearing portions 40b of the casing 40.

The frame 43 couples the lever base 42 of the bending operation lever 41 rotatably relative to the other (the central axis UD) of the predetermined central axes. In other words, the above frame 43 is provided with the plurality of (two) second bearing portions 43b (see FIG. 2) having a plurality of (two) holes (not shown; holes similar to holes corresponding to the holes 40a of the casing 40) ratably holding the plurality of (two) second rotation shafts 44 (UD), respectively.

Here, the above plurality of (two) second bearing portions 43b are formed on parts (the second end portions 43a (UD); see FIG. 2) of the frame 43 and are formed integrally with the frame 43.

On the frame 43, the above plurality of (two) second bearing portions 43b are provided at mutually facing positions on a side face of the frame 43, and the plurality of (two) holes (not shown) are provided at mutually facing positions on the side face of the frame 43. Here, the plurality of (two) holes of the plurality of (two) second bearing portions 43b are formed such that an axis connecting centers of the respective holes is coaxial with the other (the central axis UD) of the above predetermined central axes.

On the above frame 43, the second rotation shafts 44 (UD) are insertedly arranged in the above plurality of (two) holes, respectively. The second rotation shafts 44 (UD) support the lever base 42 of the bending operation lever 41 rotatably around the central axis UD in the second bearing portions 43b of the frame 43. In this case, at least parts of the second rotation shafts 44 (UD) that are in contact with inner circumferences of the holes are spherically formed. In the point, the above second rotation shafts 44 (UD) have a configuration and operation as in the first rotation shafts 44 (RL) described above.

Note that the second rotation shafts 44 (UD) are not limited to the above example in which the spherical portions 44d are formed on the second rotation shafts 44 (UD), and are also similar to the first rotation shafts 44 (RL) described above in the point that a form is also possible in which the above spherical portions are formed on at least parts on the inner circumferences of the holes, which are in contact with the outer circumferences of the rotation shafts.

A screw portion as in the above first rotation shaft 44 (RL) is formed on a part of the second rotation shaft 44 (UD) near a distal end of the second rotation shaft 44 (UD) (not shown; corresponding to the screw portion 44c). In the lever base 42, a hole (not shown; a hole similar to a hole corresponding to the hole 43d of the frame 43) is formed coaxially with the central axis UD in a manner of causing the hole to correspond to the screw portion. For the hole, a screw groove to screw onto the above screw portion is formed coaxially with the central axis UD (not shown; corresponding to the screw groove 43c). According to the configuration, the two second rotation shafts 44 (UD) are insertedly arranged in the holes, respectively, on the frame 43, and the respective screw portions of the two second rotation shafts 44 (UD) screw into the screw grooves of the lever base 42. Consequently, the lever base 42 is supported rotatably around the central axis UD by the two second rotation shafts 44 (UD) in the second bearing portions 43b of the frame 43.

The above first rotation shaft 44 (RL) has an outer diameter portion larger than an inner diameter of the hole 40a of the casing 40 and has a flange portion 44a which is a position defining portion defining a position of the first rotation shaft 44 (RL). The flange portion 44a is provided on one end portion of the first rotation shaft 44 (RL) and constitutes a part of the first rotation shaft 44 (RL). In other words, the above flange portion 44a and the first rotation shaft 44 (RL) are integrally formed. The flange portion 44a has a function of performing positioning in an axial direction of the first rotation shaft 44 (RL) by abutting a part of the casing 40.

Similarly, the above second rotation shaft 44 (UD)) has an outer diameter portion larger than an inner diameter of the hole of the frame 43 (not shown; a hole corresponding to the hole 40a of the above casing 40) and has a flange portion 44a which is a position defining portion defining a position of the second rotation shaft 44 (UD). The flange portion 44a is provided on one end portion of the second rotation shaft 44 (UD) and constitutes a part of the second rotation shaft 44 (UD). In other words, the above flange portion 44a and the second rotation shaft 44 (UD) are integrally formed. The flange portion 44a has a function of performing positioning in an axial direction of the second rotation shaft 44 (UD) by abutting a part of the casing 40.

On each of the above flange portions 44a, a jig engaged portion 44b, which is an engaged portion with which a jig (not shown; for example, a minus-driver-shaped jig) for adjusting a clearance is to engage, is formed. Note that though the example in which the jig engaged portion 44b is provided on the flange portion 44b is shown in the present embodiment, the jig engaged portion 44b is not limited to the form. The above jig engaged portion 44b is only required to have a function of causing a rotation shaft to rotate via a jig. Therefore, the jig engaged portion 44b can be provided on an end portion of the rotation shaft.

Each of the above rotation shafts (the first rotation shafts 44 (RL), the second rotation shafts 44 (UD)) is configured such that a position in a direction along a central axis (UD, RL) that the rotation shaft corresponds to is adjustable.

In other words, the screw portion 44c of the first rotation shaft 44 (RL) and the screw groove 43c of the frame 43 constitute a position adjustment mechanism configured to adjust a relative positional relationship between the first rotation shaft 44 (RL) and the frame 43 and adjust a clearance between the first bearing portion 40b and the flange portion 44a.

Similarly, the screw portion (not shown) of the second rotation shaft 44 (UD) and the screw groove (not shown) of the lever base 42 constitute a position adjustment mechanism configured to adjust a relative positional relationship between the second rotation shaft 44 (UD) and the lever base 42 and adjust a clearance between the second bearing portion 43b and the flange portion 44a.

Furthermore, on a part where the flange portion 44a of each rotation shaft and a corresponding bearing portion 40b, 43b abuts each other, a spacer member 45 formed in an almost annular shape using soft material, for example, a resin member is disposed.

In other words, the above spacer member 45 is provided between the flange portion 44a, which is a rotating part of each rotation shaft, and a part of the casing 40 or the frame 43, which is a part on a fixation side where the flange portion 44a abuts and slides.

Therefore, the above spacer member 45 prevents abrasion between the flange portion 44a of each rotation shaft and the part on the fixation side (the casing 40, the frame 43) which the flange portion 44a abuts when each rotation shaft rotates in a corresponding bearing portion 40b, 43b.

Furthermore, it is desirable to perform R chamfering processing for edge portions of each rotation shaft, the flange portions 44a and each of bearing portions 40b, 43b. By the devices as described above, abrasion among parts can be further prevented.

According to such a configuration, it is possible to, by applying a jig to the jig engaged portion 44b provided on the flange portion 44a and causing each rotation shaft to rotate in a predetermined direction in a state in which the screw portion 44c is caused to screw into the screw groove 43c, cause the rotation shaft 44 to advance or retreat in the direction along the predetermined central axis (UD, RL). Consequently, it is possible to adjust a position of each rotation shaft in the direction along the predetermined central axis (UD, RL) and adjust a clearance between each corresponding bearing portion (the first bearing portion 40b, the second bearing portion 43b) and the flange portion 44a in the direction along the predetermined central axis (UD, RL). Here, the position adjustment of the rotation shafts is performed in an assembly process.

As for the position adjustment of each rotation shaft in this case, by performing management so that contact pressure between the bearing portion (40b, 43b) and the flange portion 44a is appropriate, a frictional force of a part where both parts (the bearing portion (40b, 43b) and the flange portion 44a) slide can be controlled.

After the position adjustment of each rotation shaft is performed as described above, each rotation shaft, the frame 43 and the lever base 42 are fixed, for example, using adhesive or the like.

As described above, according to the above one embodiment, the bending operation mechanism 25 of the endoscope 2 provided with a joystick type operation member for a bending operation (the bending operation lever 41) is configured being provided with: the bending operation lever 41 with one end (the lever base 42) being rotatably held around the predetermined central axis UD relative to the predetermined central axis UD; the frame 43 with which the bending operation lever 41 is coupled, the frame 43 rotating relative to the predetermined central axis RL together with the bending operation lever 41; the rotation shafts (the first rotation shafts 44 (RL), the second rotation shafts 44 (UD)) coupled with the end portions (43a) of the frame 43 coaxially with the predetermined central axes (UD, RL); the bearing portions (the first bearing portions 40b (parts of the casing 40), the second bearing portions 43b (parts of the frame 43)) having the holes (40a and the like) rotatably holding the rotation shafts, respectively; and the flange portions 44a provided on the end portions of the respective rotation shafts and having the outer diameter portions larger than the inner diameters of the respective holes (40a and the like). In this case, by causing the screw grooves 43c of the frame 43 to screw onto the screw portions 44c of the first rotation shafts 44 (RL) and causing the screw grooves (not shown) of the lever base 42 to screw onto the screw portions (not shown) of the second rotation shafts 44 (UD) to cause each rotation shaft to advance or retreat in the direction along the predetermined central axis (UD, RL), adjustment of the position of each rotation shaft in the direction along the predetermined central axis (UD, RL) can be freely performed, and clearances between the bearing portions (40b, 43b) in the direction along the predetermined central axis (UD, RL) and the flange portions 44a can be adjusted.

In short, the respective rotation shafts are provided with the flange portions 44a, and assembly is performed so that the flange portions 44a are in contact with the bearing portions (40b, 43b). By providing the screw portion to each rotation shaft and providing the screw groove on the corresponding frame 43 and lever base 42, a configuration is made so that the positions of the respective rotation shafts on the respective central axes UD, RL (thrust directions) can be adjusted.

According to such a configuration, it is possible to, when the bending operation lever 41 rotates around the predetermined central axis (UD, RL), suppress occurrence of rotation rattling, looseness and galling, or excessive friction caused between the rotation shafts and the bearing portions (40b, 43b), and, therefore, it is possible to always realize a smooth tilting operation of the bending operation lever 41.

Each rotation shaft has the spherical portion 44d obtained by forming at least a part of the rotation shaft that is in contact with the inner circumference of the hole (40a or the like) in a spherical shape. According to the configuration, since the first rotation shaft 44 (RL) and the inner circumference of the hole 40a of the first bearing portion 40b can be in line contact with each other, it is possible to, for example, even if arrangement of the hole is slightly displaced from the central axis according to accuracy of machining, secure smooth rotation of the rotation shaft, and, therefore, a smooth tilting operation can be performed.

Note that in the form in which at least the part that is in contact with the outer circumference of the rotation shaft is formed in a spherical shape, similar effects can be obtained.

Furthermore, by causing the spacer member 45 to be interposed between the bearing portion (40b, 43b) and the flange portion 44a, the flange portion 44a rotates abutting a part on the fixation side (the casing 40, the frame 43) when each rotation shaft rotates in a corresponding bearing portion (40b, 43b), and, thereby, it is possible to prevent both of the members from being worn away.

### [Modifications]

In the bending operation mechanism 25 of the endoscope 2 of the one embodiment described above, each of the predetermined rotation shafts (44 (RL), 44 (UD)) is rotatably disposed on a predetermined part of the fixing member (the casing 40) using the bearing portion (40b, 43b).

Further, the above predetermined rotation shafts (44 (RL), 44 (UD)) are provided with the flange portions 44a and the screw portions 44c, and the screw grooves 43c are provided on the frame 43 and lever base 42 sides.

By causing the screw portion 44c to screw into the screw groove 43c, causing each of the above predetermined rotation shafts (44 (RL), 44 (UD)) to advance or retreat in the axial direction of the rotation shaft, and causing the flange portion 44a to abut the outer circumferential surface of the casing 40 (the bearing portion (40b, 43b)), the position adjustment mechanism for adjusting relative positional relationships between the above predetermined rotation shaft (44 (RL), 44 (UD)), and the frame 43 and the lever base 42 is configured.

The configuration of the above position adjustment mechanism is, however, not limited to the example shown in the one embodiment described above, but various forms are conceivable. Three modifications of the above position adjustment mechanism will be illustrated below.

A basic configuration of each modification is almost the same as in the one embodiment described above. Therefore, in the description below, description of same components is omitted, and only different parts will be described in detail.

### First Modification

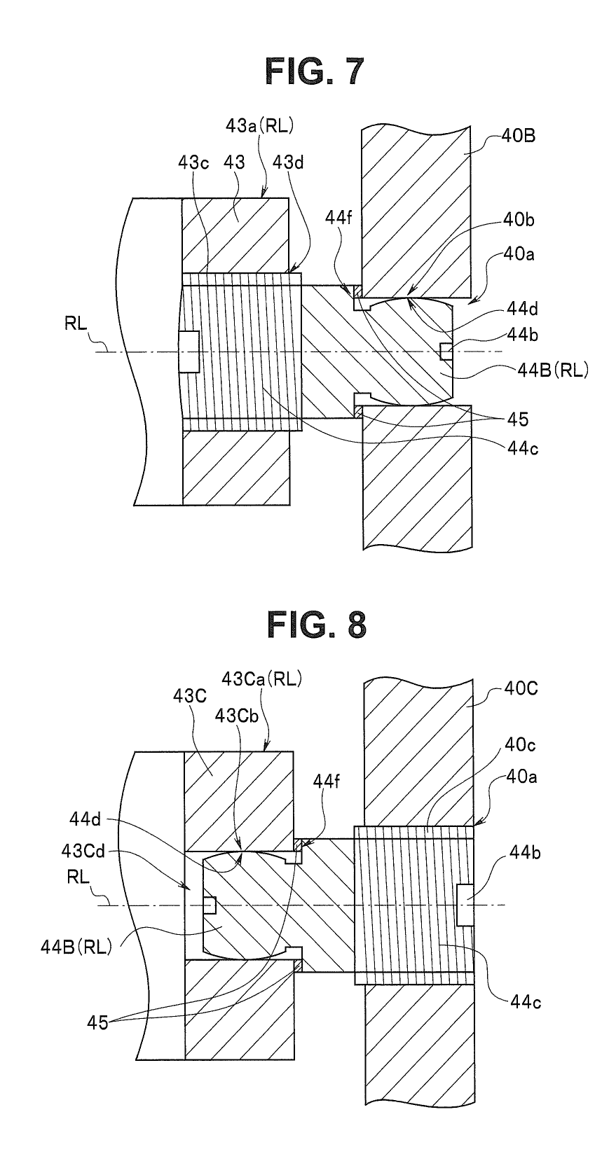

FIG. 7 is a main part enlarged cross-sectional view showing a first modification of the position adjustment mechanism configured to adjust relative positional relationships between the predetermined rotation shaft, and the frame and the lever base in the endoscope bending operation mechanism of the one embodiment of the present invention. FIG. 7 corresponds to FIG. 4 in the above one embodiment. Note that though FIG. 7 illustrates only the central axis RL, an almost the same configuration is also assumed for the central axis UD.

The first modification is different from the above one embodiment in a point that a first rotation shaft 44B (RL) is configured being provided with a step portion 44f as the position defining portion instead of the flange portion 44a described above. Therefore, shapes of the bearing portion 40b, the hole 40a and the like provided on a casing 40B are also different accordingly. Furthermore, the present modification is different in a point that the spacer member 45 is provided between the step portion 44f provided on the first rotation shaft 44B (RL) and an inner circumferential wall of the casing 40 (the bearing portion 40b).

In other words, in the configuration of the present modification, the first rotation shaft 44B (RL) is attached to a part (the first end portion 43a (RL)) of the frame 43 by causing the screw portion 44c to screw into the screw groove 43c.

In this state, a predetermined adjustment jig (not shown) is applied to the jig engaged portion 44b provided on one end (the spherical portion 44d side) of the first rotation shaft 44B (RL) and is rotated. At the time, the above first rotation shaft 44B (RL) is caused to move in the axial direction of the first rotation shaft 44B (RL) and advance to the casing 40 (the bearing portion 40b) side from an inner side of the frame 43 so that the first rotation shaft 44B (RL) abuts the casing 40 (the bearing portion 40b).

Then, the step portion 44f of the first rotation shaft 44B (RL) is caused to abut the inner circumferential wall of the casing 40 (the bearing portion 40b) via the spacer member 45. Consequently, the first rotation shaft 44B (RL) is positioned in the axial direction of the first rotation shaft 44B (RL). Therefore, consequently, position adjustment between the first rotation shaft 44B (RL) and the frame 43 is performed. Other components are similar to those of the one embodiment described above.

Effects as in the one embodiment described above can be obtained by the above first modification configured as described above.

### Second Modification

FIG. 8 is a main part enlarged cross-sectional view showing a second modification of the position adjustment mechanism configured to adjust relative positional relationships between the predetermined rotation shaft, and the frame and the lever base in the endoscope bending operation mechanism of the one embodiment of the present invention. FIG. 8 also corresponds to FIG. 4 in the above one embodiment. Note that in FIG. 8, only the central axis RL will be described (almost the same for the central axis UD).

A basic configuration of the second modification is almost the same as in the first modification described above. The present modification is different in a point that a screw groove 40c corresponding to the screw portion 44c of the first rotation shaft 44B (RL) is provided for the hole 40a on a casing 40C side.

A point is different that a configuration is made by providing a bearing portion 43Cb in a hole 43Cd on a first end portion 43Ca (RL) side, the first end portion 43Ca (RL) being a part of the frame 43C.

Note that in this case, a configuration of the first rotation shaft 44B (RL) itself is quite similar to that of the above first modification. Therefore, the same reference numeral or character of the above first modification is given, and detailed description will be omitted.

The present modification is different in a point that the spacer member 45 is provided between the step portion 44f, which is the position defining portion of the first rotation shaft 44B (RL), and an outer circumferential wall of the frame 43C (a bearing portion 40Cb).

Therefore, in the configuration of the present modification, the one end (the spherical portion 44d side) of the first rotation shaft 44B (RL) is caused to pass through a hole 40a of the casing 40C and, after that, caused to be fitted into the hole 43Cd of the frame 43C. Therefore, a diameter of the spherical portions 44d of the first rotation shaft 44B (RL) is set to be smaller than an inner diameter of the hole 40a of the casing 40C.

At the same time, the screw portion 44c of the first rotation shaft 44B (RL) is caused to screw into the screw groove 40c of the casing 40C to attach the first rotation shaft 44B (RL) to the casing 40C.

In this state, by applying a predetermined adjustment jig (not shown) to the jig engaged portion 44b of the first rotation shaft 44B (RL) and causing the adjustment jig to rotate, the above first rotation shaft 44B (RL) is caused to move in the axial direction of the first rotation shaft 44B (RL) and advance toward the frame 43C (bearing portion 43Cb) side from an outer side of the frame 43C (the casing 40C side) to cause the first rotation shaft 44B (RL) to abut the frame 43C (the bearing portion 43Cb).

Then, the step portion 44f of the first rotation shaft 44B (RL) is caused to abut the outer circumferential wall of the frame 43C (the bearing portion 40Cb) via the spacer member 45. Consequently, the first rotation shaft 44B (RL) is positioned in the axial direction of the first rotation shaft 44B (RL). Consequently, position adjustment between the first rotation shaft 44B (RL) and the frame 43 is performed. Other components are similar to those of the one embodiment described above.

Effects as in the one embodiment described above can be obtained by the above second modification configured as described above.

### Third Modification

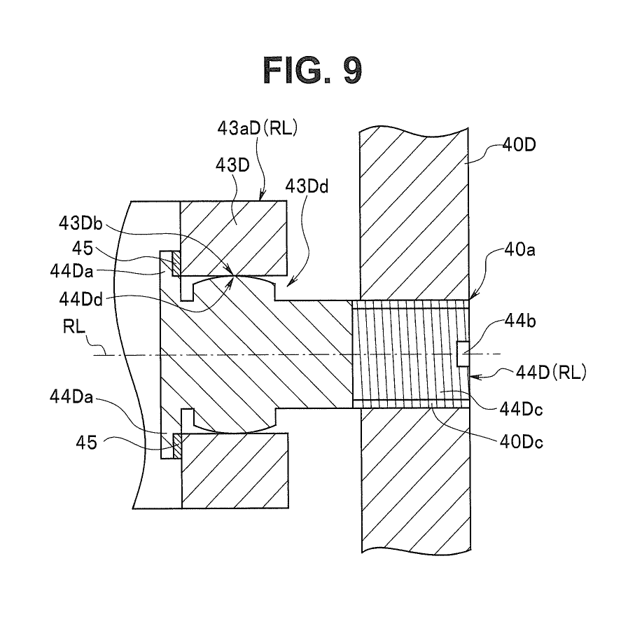

FIG. 9 is a main part enlarged cross-sectional view showing a third modification of the position adjustment mechanism configured to adjust relative positional relationships between the predetermined rotation shafts, and the frame and the lever base in the endoscope bending operation mechanism of the one embodiment of the present invention. FIG. 9 also corresponds to FIG. 4 in the above one embodiment. Note that in FIG. 9, only the central axis RL will be described (almost the same for the central axis UD).

A basic configuration of the third modification is almost the same as in the one embodiment described above and the above second modification.

In other words, the present modification is almost the same as the above one embodiment in a point that the first rotation shaft 44D (RL) is configured being provided with a flange portion 44Da and a screw portion 44Dc.

In the present modification, however, the flange portion 44Da of the first rotation shaft 44D (RL) is arranged in a manner of abutting an inner side surface of a frame 43D via the spacer member 45. A screw groove 40Dc corresponding to the screw portion 44Dc of the first rotation shaft 44D (RL) is provided for a hole 40Da on a casing 40D side almost the same as in the above second modification.

Therefore, in the present modification, a bearing portion 43Db is provided in a hole 43Dd on a part (a first end portion 43Da (RL) side) of the frame 43D.

A configuration is made so that a spherical portion 44Dd of the first rotation shaft 44D (RL) abuts an inner circumference of the hole 43Dd of the above bearing portion 43Db.

According to such a configuration, in the present modification, the other end (the screw portion 44Dc side) of the first rotation shaft 44D (RL) is caused to pass through the hole 43Dd of the frame 43D, and, after that, the screw portion 44Dc is caused to screw into the screw groove 40Dc of the hole 40Da of the casing 40D. Consequently, the first rotation shaft 44D (RL) is attached to the casing 40D. Therefore, a diameter of the screw portion 44Dc of the first rotation shaft 44D (RL) is set to be smaller than an inner diameter of the hole 43Dd of the frame 43D.

In this state, by applying a predetermined adjustment jig (not shown) to the jig engaged portion 44b of the first rotation shaft 44D (RL) and causing the adjustment jig to rotate, the above first rotation shaft 44D (RL) is caused to move in the axial direction of the first rotation shaft 44D (RL), and the flange portion 44Da is caused to abut an inner circumference of the frame 43D via the spacer member 45. Consequently, the first rotation shaft 44D (RL) is positioned in the axial direction of the first rotation shaft 44D (RL). Therefore, consequently, position adjustment between the first rotation shaft 44D (RL) and the frame 43 is performed. Other components are similar to those of the one embodiment described above.

Effects as in the one embodiment described above can be obtained by the above third modification configured as described above.

The present invention is not limited to the embodiment described above, and it is, of course, possible to make various modifications and applications within a range not departing from the spirit of the invention. Furthermore, the above embodiment includes inventions at various stages, and various inventions can be extracted by appropriately combining a plurality of disclosed constituent features. For example, even if some constituent features are deleted from all constituent features shown in the above one embodiment, a configuration obtained after deleting the constituent features can be extracted as an invention if the problem to be solved by the invention can be solved, and the advantageous effects of the invention can be obtained. Furthermore, components of different embodiments may be appropriately combined. The present invention is only limited by accompanying claims and not restricted by a particular practiced aspect of the claims.

The present invention can be applied not only to an endoscope control apparatus in a medical field but also to an endoscope control apparatus in an industrial field.

What is claimed is:

1. An endoscope bending operation mechanism comprising:
an operation lever with one end being rotatably held relative to a predetermined central axis;
a frame with which the operation lever is coupled, the frame being configured to rotate relative to the predetermined central axis with the operation lever;
a fixing member rotatably supporting the frame;
a rotation shaft coupled with an end portion of one of the frame and the fixing member coaxially with the predetermined central axis;
a bearing portion provided on the other of the frame and the fixing member and having a hole rotatably holding the rotation shaft; and
a position defining portion provided on a part of the rotation shaft, the position defining portion having an outer diameter portion larger than an inner diameter of the hole and abutting a part of the other of the frame and the fixing member to define a position of the rotation shaft in an axial direction of the rotation shaft, wherein
the rotation shaft is configured such that a position in a direction along the predetermined central axis is adjustable; and by adjusting the position of the rotation shaft in the direction along the predetermined central axis, a clearance between the bearing portion and the position defining portion in the direction along the predetermined central axis is adjusted.

2. The endoscope bending operation mechanism according to claim 1, wherein the rotation shaft and the position defining portion are integrally formed.

3. The endoscope bending operation mechanism according to claim 1, wherein
a screw groove is formed on the frame or the fixing member coaxially with the predetermined central axis;
a screw portion to screw into the screw groove is formed on the rotation shaft; and
when the clearance is adjusted, the rotation shaft is caused to advance or retreat in the direction along the predetermined central axis by causing the screw portion to screw into the screw groove.

4. The endoscope bending operation mechanism according to claim 1, wherein an engaged portion with which a jig for adjusting the clearance engages is formed on the rotation shaft.

5. The endoscope bending operation mechanism according to claim 1, wherein at least a part of the rotation shaft that is in contact with an inner circumference of the hole is formed in a spherical shape.

6. The endoscope bending operation mechanism according to claim 1, wherein at least a part of the hole that is in contact with an outer circumference of the rotation shaft is formed in a spherical shape.

7. The endoscope bending operation mechanism according to claim 1, wherein a spacer member interposes between the bearing portion and the position defining portion.

8. An endoscope bending operation mechanism comprising:
an operation lever with one end being rotatably held relative to a predetermined central axis;
a frame configured to rotatably support the operation lever;
a rotation shaft coupled with one of the frame and the operation lever coaxially with the predetermined central axis;
a bearing portion provided on the other of the frame and the operation lever and having a hole rotatably holding the rotation shaft; and
a position defining portion provided on a part of the rotation shaft, the position defining portion having an outer diameter portion larger than an inner diameter of the hole and abutting a part of the other of the frame and the operation lever to define a position of the rotation shaft in an axial direction of the rotation shaft,
wherein the rotation shaft is configured such that a position in a direction along the predetermined central axis is adjustable; and by adjusting the position of the rotation shaft in the direction along the predetermined central axis, a clearance between the bearing portion and the position defining portion in the direction along the predetermined central axis is adjusted.
